(19) United States
(12) Patent Application Publication
(10) Pub. No.: US 2005/0203533 A1
(43) Pub. Date: Sep. 15, 2005

(54) Technique and instrumentation for intervertebral prosthesis implantation

(75) Inventors: Joe W. Ferguson, Collierville, TN (US); Carlos E. Gil, Collierville, TN (US); Toney Ray Owsley, Southaven, MS (US)

Correspondence Address: HAYNES AND BOONE, LLP, 901 MAIN STREET, SUITE 3100, DALLAS, TX, 75202, US

(73) Assignee: SDGI Holdings, Inc., Wilmington, DE (US)

(21) Appl. No.: 10/799,835

(22) Filed: Mar. 12, 2004

(51) Int. Cl. A61B 17/56

(52) U.S. Cl. 606/90

(57) ABSTRACT

An assembly is used for preparing an intervertebral disc space between a pair of vertebral bodies to receive a prosthesis. The assembly comprises a distractor having a first distracting arm and a second distracting arm. The assembly further comprises a first anchoring fastener for movably coupling the first distracting arm to a first one of the vertebral bodies. The first anchoring device is able to rotate relative to the first distracting arm.

### BACKGROUND

Recently, technical advances in the design of joint reconstructive devices has revolutionized the treatment of degenerative joint disease, moving the standard of care from arthrodesis to arthroplasty. Reconstruction of a damaged joint with a functional joint prosthesis to provide motion and to reduce deterioration of the adjacent bone and adjacent joints is a desirable treatment option for many patients. For the surgeon performing the joint reconstruction, specialized instrumentation and surgical methods may be useful to facilitate precise placement of the prosthesis.

### SUMMARY

In one embodiment, an assembly is used for preparing an intervertebral disc space between a pair of vertebral bodies to receive a prosthesis. The assembly comprises a distractor having a first distracting arm and a second distracting arm. The assembly further comprises a first anchoring fastener for movably coupling the first distracting arm to a first one of the vertebral bodies. The first anchoring device is able to rotate relative to the first distracting arm.

In another embodiment, a method is disclosed for preparing an intervertebral disc space, between first and second vertebral bodies of a vertebral column, to receive an intervertebral prosthesis. The method comprises positioning first and second anchoring fasteners into the first and second vertebral bodies, respectively. The method further comprises attaching a distractor assembly to the first and second anchoring fasteners, wherein a first arm of the distractor assembly is attached to the first anchoring fastener and a second arm of the distractor assembly is attached to the second anchoring fastener. The method also comprises moving the first and second arms of the distractor, in parallel, relative to one another and rotating the first and second vertebral bodies relative to the first and second arms, respectively.

### BRIEF DESCRIPTION OF THE DRAWINGS

FIG. 1 is a sagittal view of a vertebral column having a damaged disc.

FIG. 2 is a flowchart describing a surgical technique.

FIG. 3 is an drilling assembly according to one embodiment of the current disclosure.

FIG. 4 is an isometric view of an anchor post according to one embodiment of the current disclosure.

FIG. 5 is a distractor assembly according to a one embodiment of the current disclosure.

FIG. 6 is cross-sectional view of the distractor of the first embodiment shown in FIG. 5.

FIG. 7 is an exploded view of an instrumentation guide according to one embodiment of the current disclosure.

FIG. 8 is an isometric view of the instrumentation guide of FIG. 7 assembled with the distractor assembly of FIG. 4.

FIG. 9 is an assembled view of a measurement instrument according to an embodiment of this disclosure.

FIG. 10 is an assembled view of the devices of FIGS. 4, 7, and 9.

FIG. 11 is an exploded view of a cutting assembly according to one embodiment of the current disclosure.

FIG. 12 is an assembled view of the devices of FIGS. 4, 7, and 11.

### DETAILED DESCRIPTION

The present disclosure relates generally to the field of orthopedic surgery, and more particularly to instrumentation and methods for vertebral reconstruction using an intervertebral prosthesis. For the purposes of promoting an understanding of the principles of the invention, reference will now be made to embodiments or examples illustrated in the drawings, and specific language will be used to describe the same. It will nevertheless be understood that no limitation of the scope of the invention is thereby intended. Any alteration and further modifications in the described embodiments, and any further applications of the principles of the invention as described herein are contemplated as would normally occur to one skilled in the art to which the invention relates.

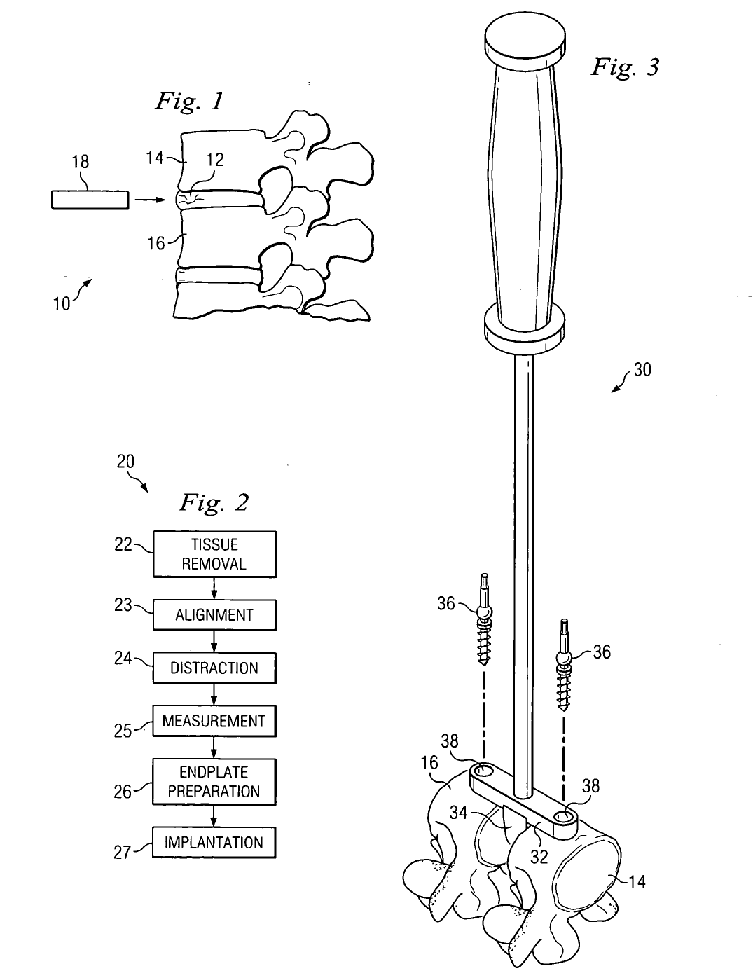

Referring first to FIG. 1, the numeral 10 refers to a human anatomy having a joint location which in this example includes an injured, diseased, or otherwise damaged intervertebral disc 12 extending between vertebrae 14, 16. The damaged disc may be replaced by an intervertebral disc prosthesis 18 which may be a variety of devices including the prostheses which have been described in U.S. Pat. Nos. 5,674,296; 5,865,846; 6,156,067; 6,001,130 and in U.S. patent application Nos. 2002/0035400; 2002/0128715; and 2003/0135277 which are incorporated by reference herein.

A surgical technique for repairing the damaged joint may be represented, in one embodiment, by the flowchart 20 depicted in FIG. 2. Referring first to step 22, all or a portion of the damaged disc 12 may be excised. This procedure may be performed using an anterior, anterolateral, lateral, or other approach known to one skilled in the art, however, the forgoing embodiments will be directed toward a generally anterior approach. Generally, the tissue removal procedure 22 may include positioning and stabilizing the patient. Fluoroscopic or other imaging methods may be used to assist with vertebral alignment and surgical guidance. Imaging techniques may also be used to determine the proper sizing of the intervertebral prosthesis 18. In one embodiment, a sizing template may be used to pre-operatively determine the correct prosthesis size. The tissue surrounding the disc space may be retracted to access and verify the target disc space. The area of the target disc may be prepared by removing excess bone, including osteophytes which may have developed, and other tissues which may include portions of the annulus and all or portions of the nucleus pulpous. The tissue removal procedure 22, which may include a discectomy procedure, may alternatively or additionally be performed after alignment and/or measurement procedures have been taken.

Proceeding to step 23 of FIG. 2, various alignment procedures may be conducted to align the intervertebral space in preparation for the disc prosthesis 18. The transverse center of the disc space may be determined and marked. Next, an alignment guide 30, which may include a drill guide 32 and an intervertebral portion 34, is used to determine the accurate placement of anchoring fasteners 36. The drill guide 32 may have apertures 38 for guiding a drill.

The intervertebral portion 34, attached to the drill guide 32, may be inserted between the adjacent vertebral bodies 14, 16. The drill guide 32 may be used as a template to mark the position for the anchoring fasteners 36, for example by pre-drilling into the vertebral bodies 14, 16. Using the drill guide 32 as a template may facilitate accurate placement of the anchoring fasteners 36 and later attached instrumentation with respect to the endplates of vertebral bodies 14. For example the marked positions may be located a generally fixed distance from the center of the disc space. After the locations have been marked, the anchoring fasteners 36 may then be inserted at the locations determined by the drill guide 32.

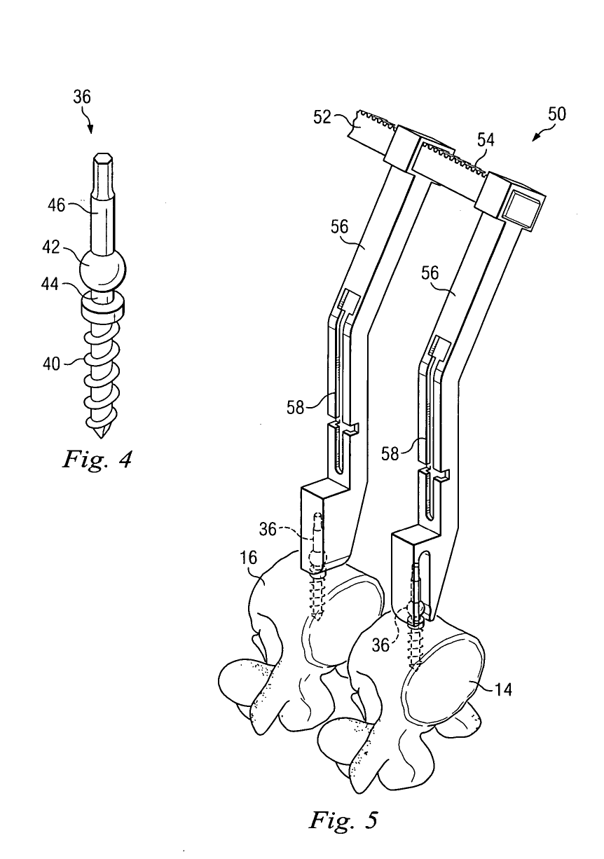

As shown in detail in FIG. 4, in this embodiment, the anchoring fastener 36 may have a threaded portion 40 and a partially spherical portion 42. A distance maintenance portion 44 may extend between the threaded and partially spherical portions 40, 42. An elongated portion 46 may extend from the partially spherical portion 42.

Proceeding now to step 24 of FIG. 2, the vertebral bodies 14, 16 may be distracted and held in tension in preparation for further processing of the intervertebral disc space. Referring now to FIG. 5, the distraction of step 24 (FIG. 2) may be accomplished with a distractor assembly 50 attached to the anchoring fasteners 36. The distractor assembly 50 may include a cross bar member 52 having a securing mechanism 54. A pair of distracting arms 56 may be attached to the cross bar member. In the embodiment of FIG. 5, the securing mechanism 54 is a ratchet system which can maintain a selected distance between the distracting arms 56. A variety of alternative securing mechanisms 54 including clamps, threaded connectors, and pins may be selected as a means to maintain a selected distance between the distracting arms 56. At least one of the distracting arms 56 may be movably connected to cross bar member 52 by the securing mechanism 54. Each of the distracting arms 56 may include attachment guides 58 such as the t-slots shown in FIG. 5. Other types of attachment guides may include dove tailed grooves or other mechanical connectors. The attachment guides 58 may be used to locate, hold, and guide measuring and cutting instrumentation as will be described below. The attachment guides 58 may include stops or other features useful for position verification or instrument support.

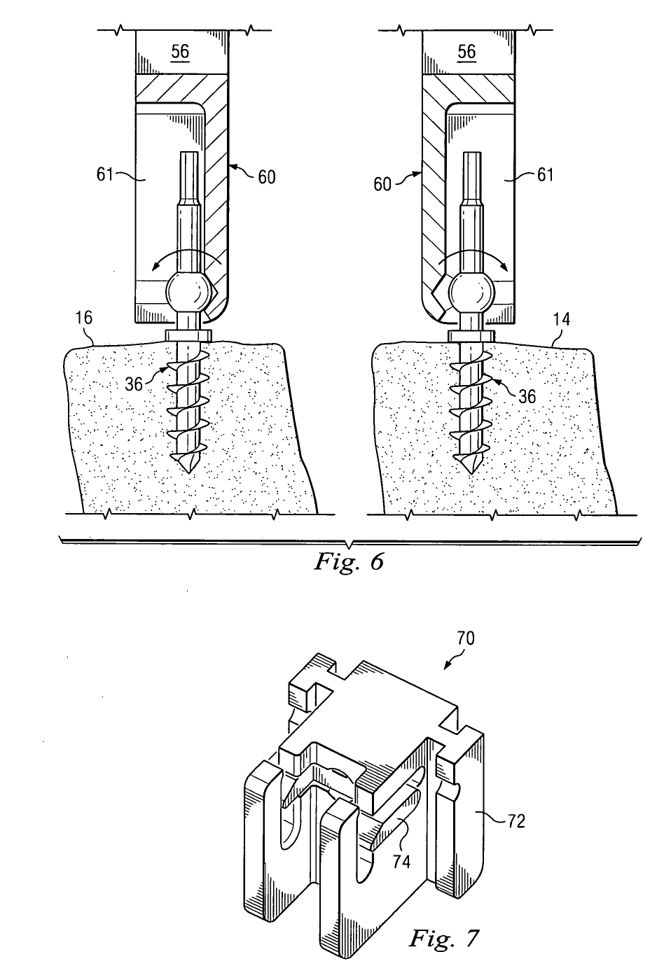

Referring now to FIG. 6, the distracting arms 56 may further include end portions 60 which connect to the anchoring fasteners 36. The end portions 60 may be integrally formed with the distracting arms 56 or may be movably or fixedly connected to the distracting arms 56. The end portions 60 may include elongated slots 61 which allow the spherical portions 42 of the anchoring fasteners 36 to slide into the end portions 60. The distance maintenance portions 44 of the anchoring fasteners 36 may be sized to constrain, to the extent desired, the axial movement (along the axis of the anchoring fasteners 36) of the distracting arms 56 relative to the surface of the vertebral bodies 14, 16. The width of the elongated slots 61 may be sized to constrain lateral movement of the anchoring fasteners 36 and the attached vertebral bodies 14, 16.

As the vertebral bodies 14, 16 are being distracted with the distractor assembly 50, the distracting arms 56 may remain relatively parallel while at least limited rotation of the vertebral bodies 14, 16 is permitted. This rotation, which may be in the sagittal plane, may occur as the spherical portion 42 rotates and/or translates in the elongated slots 61 relative to the distracting arms 56. The distance maintenance portion 44 of the anchoring fasteners 36 and the depth of the elongated slots 61 may provide limitation to the amount of rotation permitted. As the vertebral bodies 14, 16 and the anchoring fasteners 36 rotate or pivot, the anchoring fasteners 36 may remain fixed relative to the vertebral bodies 14, 16.

Referring again to FIG. 2, with the vertebral bodies 14, 16 distracted and the desired vertebral constraint applied, the surgical technique 20 proceeds to step 25. At step 25, measurements, such as a depth measurement, may be performed at the disc site to determine the proper sizing of instrumentation and devices to be used throughout the remainder of the surgical technique 20.

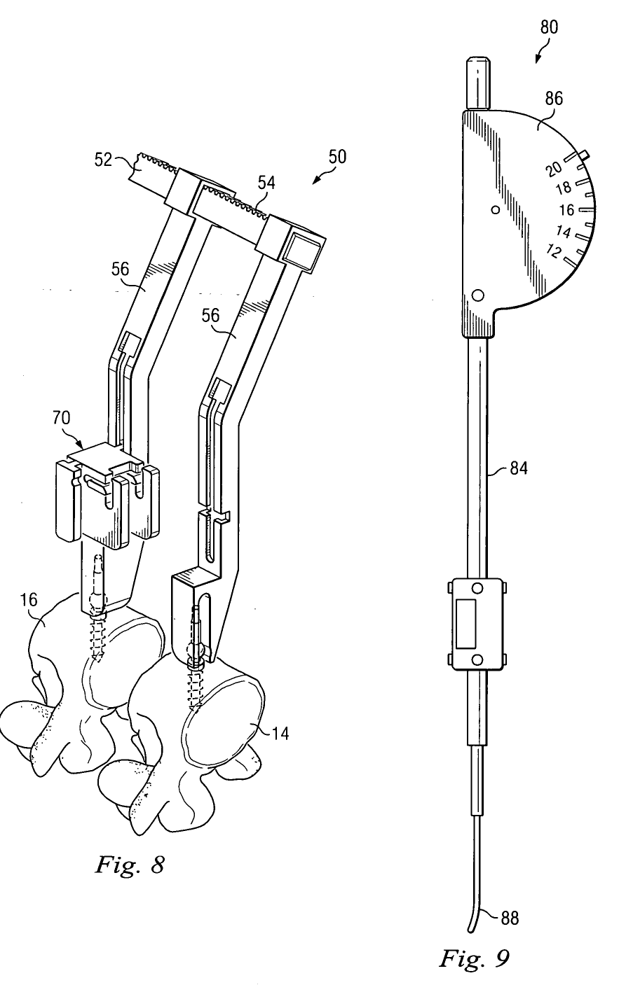

Referring now to FIGS. 7 and 8, the measurement step 24 (FIG. 2) may use a variety of instrumentation including, for example, an instrumentation guide 70 which may be attached to either of the distracting arms 56. The instrumentation guide 70, which in this embodiment includes a guide housing 72 and a retention assembly 74, may attach to the selected distracting arm 56 via the attachment guide 58 (FIG. 8). The retention assembly 74 may be used to movably or fixedly secure instrumentation, such as a measuring or a cutting instrument (as will be described below), to the guide housing 72 and thus, indirectly to the distracting arm 56. A variety of retention assemblies 74 may be used with the instrumentation guide 70 including a spring-loaded pin, clamps, threaded connectors, or other types of fasteners.

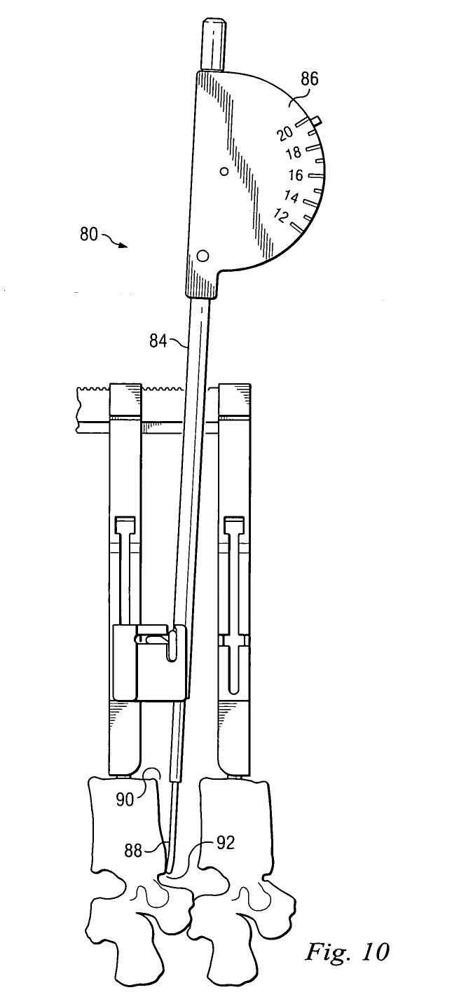

Referring now to FIGS. 9-10, a measurement instrument 80 may be used for selecting appropriately sized tools to perform subsequent operations such as endplate preparation. In this embodiment, the measurement instrument 80, which includes a shaft 82 extending between an indicator portion 86 and a probe portion 88, may movably or fixedly fasten to the instrumentation guide 70. The probe portion 88 may travel through the intervertebral disc space to provide a depth measurement. In this embodiment, the indicator portion 86 may indicate the distance from a point, such as an anterior edge 90 of the intervertebral disc space to the posterior margin 92 of the disc space. The indicator portion 86 may indicate the distance traveled by the probe portion 88 providing a measurement which can be used to determine the proper sizing of subsequently used instruments or prosthetic devices.

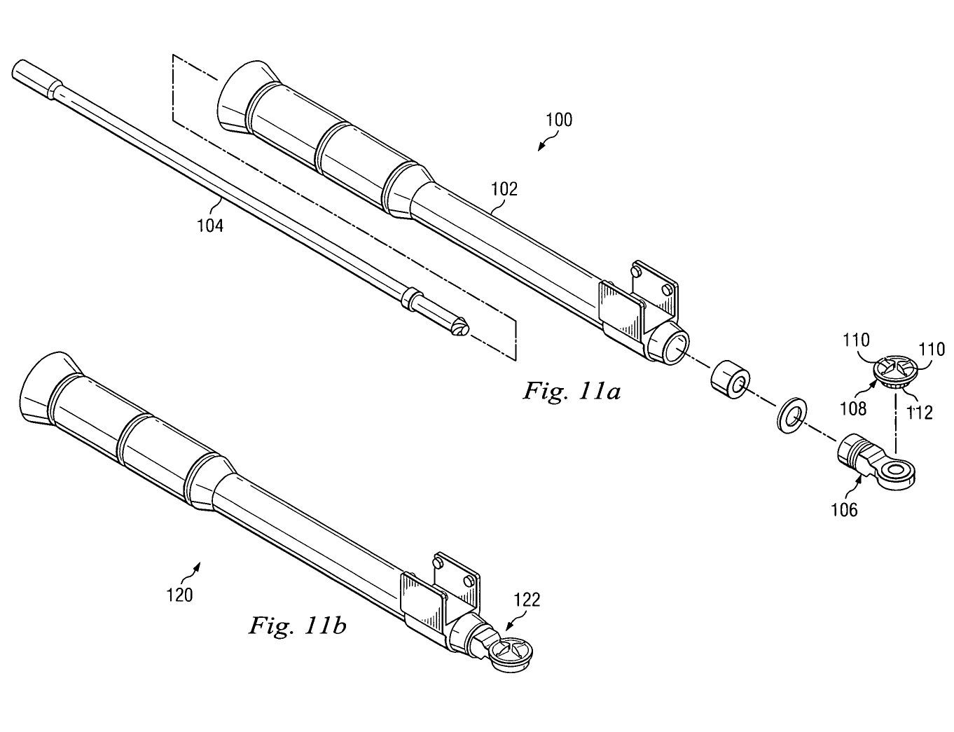

Referring again to FIG. 2, the surgical technique 20 proceeds to step 26 for further preparation of the vertebral endplate surfaces. Referring now to FIGS. 11a-11b, to prepare the endplate surfaces to provide a secure seat for the intervertebral prosthesis 18, a cutting instrument may be provided. In the embodiment of FIG. 11a, the cutting instrument 100 may comprise several component parts including an exterior shaft portion 102, an internal shaft portion 104, a cutting head 106, and a cutting device 108. The internal shaft portion 104 may extend through the exterior shaft portion 102 to engage the cutting head 106. The cutting device 108 may be attached to the cutting head 106. The cutting device 108 may have an abrasive surface 110 which can include blades, teeth, a roughened coating or any other surface capable of cutting, abrading, or milling the vertebral endplates. The cutting device 108 or cutting surface 110 may be shaped such that the profile that it creates in the vertebral endplate matches the profile of the selected intervertebral prosthesis 18 to create a secure seat for the prosthesis. The cutting instrument 100 may include a variety of other components (not shown) such as rivets, bearings, gears, and springs which may be used to assemble the components 102-108 to each other and provide movement to the cutting device 108.

In one embodiment, such as is shown in FIG. 11b, the components 102-108 of the cutting instrument 100 may be constructed to simplify cleaning, promote sterility, enhance reliability, and shorten assembly and surgical time. In one embodiment, the cutting head 106 may be a single piece of molded polymer. In this embodiment, the use of bearings and other components capable of corrosion or susceptible to wearing out easily may be reduced or eliminated. The cutting head 106 may be disposable which can simplify the cleaning of the cutting instrument 100 and may promote sterility in the surgical field. The internal shaft portion 102, which may include an integrated pinion gear, may be disposable to minimize wear on other sensitive components such as gear trains, increasing the reliability of the instrument 100. The use of a pinion shaft as the internal shaft portion 102 may also eliminate bearings and other drive train components which improves reliability and simplifies cleaning of the cutting instrument 100. The cutting device 108 may be a one-piece metal injection molded cutter having the cutting surface 110 formed on one side and gear teeth 112 integrated into the opposite side to minimize the profile. This integrated embodiment of the cutting device 108 may also promote reliability and sterility.

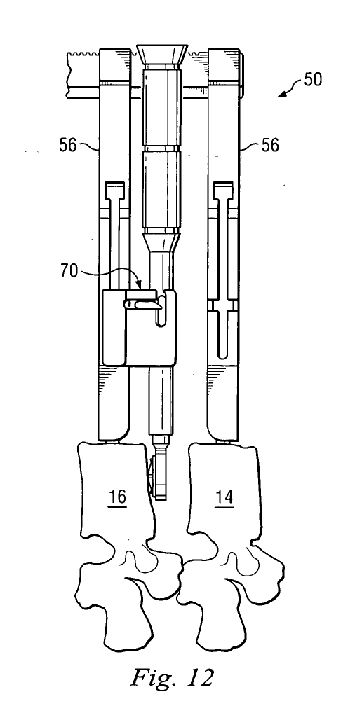

Referring now to FIG. 12, based upon the measurements taken in step 25 and the prosthesis 18 to be implanted, a cutting device 108 may be selected. The cutting instrument 100 may be assembled, as described above, using the selected cutting device 108. The instrumentation guide 70 may be attached to one of the distracting arms 56, and the cutting instrument 110 may be mounted to the instrumentation guide 70 such that the cutting device 108 is positioned adjacent to one of the vertebral endplates 16. The proper positioning of the cutting device 108 may be established with known offsets and may be verified with fluoroscopic or other imaging techniques. A power source (not shown) may be connected to the cutting instrument 100 and activated to drive the cutting surface 110 as the cutting surface shapes the selected vertebral endplate. After the first endplate surface is prepared, the instrumentation guide 70 may be moved to the second distracting arm 56 and the cutting surface 110 may be positioned adjacent to the second vertebral body 14. The cutting instrument may then be activated to shape the second vertebral body 14.

The cutting instrument described above for FIG. 11a is merely one embodiment which may be used with the distractor assembly 50 and instrumentation guide 70. In alternative embodiments, the cutting instrument maybe include a burr or other cutting devices known in the art. The cutting instrument may also include a telescoping shaft to permit lengthening of the cutting instrument. In some embodiments such as FIG. 11b, the cutting instrument 120 may be comprised largely of reusable components capable of being sterilized, such as by an autoclave. In this embodiment, a cutting head 122 may have a higher profile to accommodate a press-fit gear and other gear train components.

Referring again to FIG. 2, after the vertebral endplates are prepared, the cutting instrument 100 or 120 may be removed from the instrumentation guide 70 in preparation for implanting the intervertebral prosthesis 18 at step 27. With the cutting instrumentation removed, the intervertebral prosthesis 18 may be inserted into the prepared space using any of a variety of insertion methods. In some embodiments, the instrumentation guide 70 may be used to guide prosthesis insertion instrumentation. After the prosthesis 18 is implanted, the tension on the distractor assembly 50 may be released and the assembly 50 may be removed. The anchoring fasteners 36 may be removed and the wound closed.

Although only a few exemplary embodiments of this invention have beendescribed in detail above, those skilled in the art will readily appreciate that many modifications are possible in the exemplary embodiments without materially departing from the novel teachings and advantages of this invention. Accordingly, all such modifications are intended to be included within the scope of this invention as defined in the following claims. In the claims, means-plus-function clauses are intended to cover the structures described herein as performing the recited function and not only structural equivalents, but also equivalent structures.

1. An assembly for preparing an intervertebral disc space between a pair of vertebral bodies to receive a prosthesis, the assembly comprising:
a distractor, wherein the distractor comprises a first distracting arm and a second distracting arm; and
a first anchoring fastener for movably coupling the first distracting arm to a first one of the vertebral bodies,
wherein the first anchoring device is rotatable relative to the first distracting arm.

2. The assembly of claim 1 further comprising a second anchoring device for movably coupling the second distracting arm to a second one of the vertebral bodies.

3. The assembly of claim 1 wherein the first and second distracting arms are movable in a parallel relationship, wherein the first anchoring fastener rotatably couples the first distracting arm to the first vertebral body, and further wherein the first vertebral body rotates relative to the first distracting arm as the first and second distracting arms move in a parallel relationship.

4. The assembly of claim 1 further comprising an instrumentation guide attached to the first distracting arm.

5. The assembly of claim 4 wherein the first distracting arm comprises an attachment guide, wherein the instrumentation guide is attached to the first distracting arm by the attachment guide.

6. The assembly of claim 5 wherein the attachment guide is a mechanical connector on the first distracting arm.

7. The assembly of claim 4 further comprising a measurement instrument attached to the instrumentation guide.

8. The assembly of claim 4 further comprising a shaping instrument attached to the instrumentation guide.

9. The assembly of claim 8 wherein the attachment of the shaping instrument to the instrumentation guide is adjustable as the first vertebral body rotates relative to the first distracting arm.

10. The instrumentation guide of claim 8 further comprising a spring-loaded retention assembly for holding the shaping instrument to the instrumentation guide.

11. The assembly of claim 1 wherein the first anchoring fastener comprises a partially spherical portion.

12. The assembly of claim 11 wherein the first distracting arm comprises an elongated slot, wherein the first anchoring fastener engages the elongated slo, and further wherein the spherical portion rotates in the elongated slot relative to the first distracting arm.

13. A method for preparing an intervertebral disc space, between first and second vertebral bodies of a vertebral column, to receive an intervertebral prosthesis, the method comprising:
positioning first and second anchoring fasteners into the first and second vertebral bodies, respectively;
attaching a distractor assembly to the first and second anchoring fasteners, wherein a first arm of the distractor assembly is attached to the first anchoring fastener and a second arm of the distractor assembly is attached to the second anchoring fastener;
moving the first and second arms of the distractor, in parallel, relative to one another;
rotating the first and second vertebral bodies relative to the first and second arms, respectively.

14. The method of claim 13 further comprising shaping an first endplate of the first vertebral body independently of shaping a second endplate of a second vertebral body.

15. The method of claim 14 further comprising attaching a shaping instrument to the first distractor arm prior to shaping the first endplate.

16. The method of claim 13 wherein the first anchoring fastener is pivotable within an elongated slot in the first distractor arm.

17. The method of claim 13 wherein the positioning of the first and second anchoring fasteners is in a sagittal plane.

18. The method of claim 17 wherein the rotation of the first and second vertebral bodies is in the sagittal plane.

19. The method of claim 13 wherein the positioning of the first and second anchoring fasteners is equidistant from the center of the intervertebral disc space.

20. The method of claim 13 wherein attaching the distractor assembly to the first and second anchoring fasteners comprises sliding the first anchoring fastener into a first slot located in the first distractor arm.

21. The method of claim 13 wherein the first slot prevents transverse motion of the first vertebral body with respect to the first distractor arm.

22. An assembly for preparing an intervertebral disc space between first and second vertebral bodies to receive a prosthesis, the assembly comprising:
a distractor, wherein the distractor comprises a first distracting arm in parallel relation to a second distracting arm;
a first anchoring fastener extending between the first distracting arm and the first vertebral body, wherein the first anchoring fastener comprises a first partially spherical portion and the first distracting arm comprises a first slot and further wherein the first partially spherical portion pivotally engages the first slot; and
a second anchoring fastener extending between the second distracting arm and the second vertebral body, wherein the second anchoring fastener comprises a second partially spherical portion and the second distracting arm comprises a second slot and further wherein the second partially spherical portion pivotally engages the second slot.

23. The assembly of claim 22 further comprising an instrumentation guide attached to the first distracting arm.

24. The assembly of claim 23 further comprising a milling instrument pivotally attached to the instrumentation guide.
